(19) United States
(12) Patent Application Publication
(10) Pub. No.: US 2022/0305170 A1
(43) Pub. Date: Sep. 29, 2022

(54) WOUND-COVERING MATERIAL AND PRODUCTION METHOD THEREFOR

(71) Applicants: OJl HOLDINGS CORPORATION, Tokyo (JP); KURASHIKI BOSEKI KABUSHIKI KAISHA, Okayama (JP)

(72) Inventors: Rina TANAKA, Yokohama-shi, Kanagawa-ken (JP); Chinatsu MIYAZAKI, Neyagawa, Osaka (JP); Yuko TANAKA, Neyagawa, Osaka (JP); Minoru SUGIYAMA, Neyagawa, Osaka (JP); Kunihiro OHSHIMA, Neyagawa, Osaka (JP)

(73) Assignee: OJl HOLDINGS CORPORATION, Tokyo (JP)

(21) Appl. No.: 17/597,167

(22) PCT Filed: Jul. 1, 2020
(86) PCT No.: PCT/JP2020/025895

§ 371 Date: Dec. 28, 2021

(30) Foreign Application Priority Data
Jul. 5, 2019 (JP) 2019-126141

(51) Int. Cl. A61L 15/60; A61L 26/00; A61L 15/24; A61L 15/28

(52) U.S. Cl. CPC A61L 15/60; A61L 26/008; A61L 15/24; A61L 15/28

(57) ABSTRACT

The present invention relates to a wound covering material containing at least a hydrogel, wherein the hydrogel contains fine fibrous celluloses having ionic substituents. The present invention also relates to a method of producing the wound covering material, including a process of obtaining a hydrogel using a mixture of hydrophilic polymers and fine fibrous celluloses having ionic substituents. Accordingly, there are provided a wound covering material having excellent water retention and strength and favorable adhesiveness and peelability with respect to a living body, and a method of producing the same.

### TECHNICAL FIELD

The present invention relates to a wound covering material containing a hydrogel and a method of producing the same. Specifically, the present invention relates to a wound covering material having excellent water retention and strength and favorable adhesiveness and peelability with respect to a living body, and a method of producing the same.

### BACKGROUND ART

A hydrogel is suitably used as a wound covering material because it easily absorbs water, has high water retention, and has excellent adhesion with respect to a living body. For example, Patent Literature 1 proposes a hydrogel wound covering material including an adhesive layer made of an adhesive polyvinyl alcohol hydrogel and a water absorbing and supporting layer made of a polyvinyl alcohol hydrogel. In addition, Patent Literature 2 proposes use of a low elution hydrogel containing hydrophilic polymers, water and a quaternary ammonium compound as a wound covering material.

### PATENT LITERATURES

Patent Literature 1: Japanese Unexamined Patent Application Publication No. H9-262249

Patent Literature 2: Japanese Unexamined Patent Application Publication No. 2014-97255

### SUMMARY OF THE INVENTION

In the case of the hydrogels described in Patent Literatures 1 and 2, it is desirable to further improve a water retention capacity and increase the strength. In addition, the wound covering material is required to be easily adhered to a living body, and is also required to be easily peeled off, that is, to have peelability, and thus there is a need to improve peelability of the hydrogels described in Patent Literatures 1 and 2.

In order to address the problems in the related art, the present invention provides a wound covering material having excellent water retention and strength and favorable adhesiveness and peelability with respect to a living body and a method of producing the same.

The present invention relates to a wound covering material containing at least a hydrogel, wherein the hydrogel contains fine fibrous celluloses having ionic substituents.

The present invention also provides a method of producing a wound covering material containing at least a hydrogel, including a process of obtaining a hydrogel using a hydrogel composition containing hydrophilic polymers and fine fibrous celluloses having ionic substituents.

The present invention can provide a wound covering material having excellent water retention and strength and favorable adhesiveness and peelability with respect to a living body.

In addition, according to the production method of the present invention, it is possible to obtain a wound covering material having excellent water retention and strength and favorable adhesiveness and peelability with respect to a living body.

### BRIEF DESCRIPTION OF THE DRAWINGS

FIG. 1 is a graph showing the relationship between a NaOH dropping amount and a pH of a dispersion solution containing fine fibrous celluloses having a phosphorus oxoacid group as an ionic substituent.

FIG. 2 is a graph showing the relationship between a NaOH dropping amount and a pH of a dispersion solution containing fine fibrous celluloses having carboxyl groups as ionic substituents.

FIG. 3 is a graph showing changes in the body weight of rats in a wound healing test.

FIG. 4 is a graph showing changes in the area ratio of wound areas of rats in a wound healing test.

### MODES FOR CARRYING OUT THE INVENTION

The inventors of the present invention have conducted extensive studies in order to address the problems in the related art described above, and as a result, found that a wound covering material using a hydrogel containing fine fibrous celluloses having an ionic substituent can maintain a moist environment suitable for wound healing due to increased water retention, and thus can promote wound healing. In addition, they found that, when the wound covering material using the hydrogel contains the fine fibrous celluloses having ionic substituents, the strength increases, and therefore it is hard to break, and handling properties, cutting properties and the like are favorable. In addition, they found that the wound covering material using the hydrogel can achieve both ease of adhesiveness and peelability with respect to skin. In the following, unless otherwise specified, “fine fibrous cellulose” is “fine fibrous cellulose having an ionic substituent.”

In one or more embodiments of the present invention, the fine fibrous celluloses may have an average fiber width of 1,000 nm or less. In addition, the fine fibrous celluloses are not particularly limited, and for example, the average fiber width is preferably 2 nm or more. When the fine fibrous celluloses have an average fiber width of less than 2 nm, since they are dissolved as cellulose molecules in water, the strength and peelability of the hydrogel are unlikely to be improved. In consideration of ease of dispersion in hydrophilic polymers, and excellent water retention and transparency, the average fiber width of the fine fibrous celluloses having ionic substituents is preferably 100 nm or less, more preferably 50 nm or less, still more preferably 20 nm or less, yet more preferably 10 nm or less, and particularly preferably 7 nm or less. When the fine fibrous celluloses having ionic substituents are observed under an electron microscope, the fiber width can be measured. Specifically, the fiber width is measured as follows.

<Fiber Width of Fine Fibrous Cellulose>

An aqueous suspension containing fine fibrous celluloses having a concentration of 0.05 mass % or more and 0.1 mass % or less is prepared, and this suspension is cast onto a hydrophilized carbon film-coated grid to prepare a TEM observation sample. If it contains fibers having a wide width, the suspension may be cast on glass and an SEM image on the surface of the cast film may be observed. Observation with an electron microscope image is performed at any magnification of 1,000, 5,000, 10,000 and 50,000 depending on the widths of the constituent fibers. However, the sample, observation conditions, and the magnification are adjusted to satisfy the following conditions.

(1) A straight line X is drawn at an arbitrary position in an observation image, and 20 or more fibers intersect the straight line X.
(2) A straight line Y that intersects the straight line perpendicularly is drawn in the same image, and 20 or more fibers intersect the straight line Y.

The widths of the fibers intersecting the straight line X and the straight line Y are visually read from the observation image that satisfies the conditions. In this manner, at least three sets of images of non-overlapping surface parts are observed, and the widths of the fibers intersecting the straight line X and the straight line Y are read from each image. Thereby, the fiber widths of at least 20 fibers×2×3=120 fibers are read. Then, the average value of the read fiber widths is used as an average fiber width of the fine fibrous celluloses.

The fiber length of the fine fibrous cellulose is not particularly limited, and for example, is preferably 0.1 μm or more and 1,000 μm or less, more preferably 0.1 μm or more and 800 μm or less, and still more preferably 0.1 μm or more and 600 μm or less. When the fiber length is set to be within the above range, it is possible to inhibit destruction of a crystalline region of the fine fibrous cellulose. In addition, it is possible to set the viscosity of the dispersion solution containing fine fibrous celluloses to be within an appropriate range. Here, the fiber length of the fine fibrous cellulose can be obtained by, for example, image analysis using TEM, SEM, or AFM.

The fine fibrous cellulose preferably has a type I crystal structure. Here, the fact that the fine fibrous cellulose has a type I crystal structure can be identified in the diffraction profile obtained from a wide angle X-ray diffraction image using CuKα (λ=1.5418 Å) monochromatic with graphite. Specifically, it can be identified that, when 20 is around 14° or more and 17° or less, two positions at which 20 is around 22° or more and 23° or less have typical peaks. The proportion of the type I crystal structure in the fine fibrous cellulose is, for example, preferably 30% or more, more preferably 40% or more, and still more preferably 50% or more. Therefore, better performance can be expected in consideration of heat resistance and exhibition of a low coefficient of thermal expansion. The degree of crystallization is determined by a general method in which an X-ray diffraction profile is measured, and its pattern is used (Seagal et al., Textile Research Journal, vol. 29, p. 786, 1959).

The axial ratio (fiber length/fiber width) of the fine fibrous cellulose is not particularly limited, and is, for example, preferably 20 or more and 10,000 or less, and more preferably 50 or more and 1,000 or less. When the axial ratio is set to the lower limit value or more, it is easy to form a hydrogel containing fine fibrous celluloses. In addition, it is easy to obtain sufficient enhanced viscosity when a solvent dispersion is prepared. If the axial ratio is set to the upper limit value or less, this is preferable because handling such as dilution becomes easy, for example, when the fine fibrous cellulose is treated as a water dispersion solution.

In one or more embodiments of the present invention, the fine fibrous cellulose has, for example, both a crystalline region and a non-crystalline region. In particular, a fine fibrous cellulose having both a crystalline region and a non-crystalline region and having a high axial ratio can be realized by a method of producing fine fibrous celluloses to be described below.

In one or more embodiments of the present invention, fine fibrous celluloses have ionic substituents. When the fine fibrous celluloses have ionic substituents, the dispersibility of the fine fibrous celluloses in the dispersion medium can be improved, and the defibration efficiency in the defibration treatment can be improved. In addition, fine fibrous celluloses can easily maintain a form of a single fiber, the dispersibility in hydrophilic polymers can be improved, the strength and water retention of the hydrogel can be improved, and both the adhesiveness and peelability with respect to skin can be achieved. The ionic substituent may include, for example, either or both of an anionic group and a cationic group.

In one or more embodiments of the present invention, an anionic group is preferable as the ionic substituent because it is easy to maintain a stable form of a single fiber even if the average fiber width of the fine fibrous celluloses is small. The anionic group is, for example, preferably at least one selected from the group consisting of a phosphorus oxoacid group or a substituent derived from a phosphorus oxoacid group (hereinafter simply referred to as a phosphorus oxoacid group), a carboxyl group or a substituent derived from a carboxyl group (hereinafter simply referred to as a carboxyl group), and a sulfur oxoacid group or a substituent derived from a sulfur oxoacid group (simply referred to as a sulfur oxoacid group). Among these, at least one selected from the group consisting of a phosphorus oxoacid group and a substituent derived from a phosphorus oxoacid group is more preferable, and a phosphoric acid group is still more preferable.

The phosphorus oxoacid group is a group in which a hydroxy group and an oxo group are bonded to a phosphorus atom, and examples thereof include a phosphoric acid group obtained by removing a hydroxy group from a phosphoric acid, and a phosphite group obtained by removing a hydroxy group from phosphorous acid (phosphonic acid group). Substituents derived from a phosphorus oxoacid group include substituents such as a phosphorus oxoacid group salt, a phosphoric acid ester group, a phosphite group salt, and a phosphite ester group. Here, substituents derived from a phosphorus oxoacid group may be contained in fine fibrous celluloses as a group in which phosphoric acid groups are condensed (for example, a pyrophosphoric acid group). A phosphorus oxoacid group or a substituent derived from a phosphorus oxoacid group can be represented by, for example, the following Chemical Formula (1).

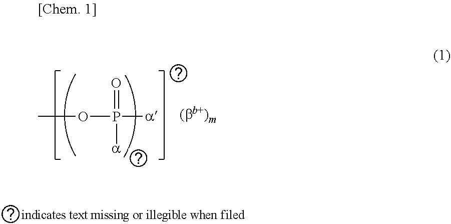

In Chemical Formula (1), a, b and n are natural numbers (where a=b×m). Of α¹, α², . . . , αⁿ and α′, a are O—, and the rest are R or OR. Here, both of αⁿ and α′ may be O—. Each R is a hydrogen atom, a saturated-linear hydrocarbon group, a saturated-branched chain hydrocarbon group, a saturated-cyclic hydrocarbon group, an unsaturated-linear hydrocarbon group, an unsaturated-branched chain hydrocarbon group, an unsaturated-cyclic hydrocarbon group, an aromatic group, or a group derived therefrom. In addition, n is preferably 1. Here, when R is a hydrogen atom, the substituent represented by Chemical Formula (1) corresponds to a phosphorus oxoacid group, and in other cases, the substituent represented by Chemical Formula (1) corresponds to a substituent derived from a phosphorus oxoacid group.

The saturated-linear hydrocarbon group is not particularly limited, and examples thereof include a methyl group, an ethyl group, an n-propyl group, and an n-butyl group. The saturated-branched chain hydrocarbon group is not particularly limited, and examples thereof include an i-propyl group and a t-butyl group. The saturated-cyclic hydrocarbon group is not particularly limited, and examples thereof include a cyclopentyl group and a cyclohexyl group. The unsaturated-linear hydrocarbon group is not particularly limited, and examples thereof include a vinyl group and an allyl group. The unsaturated-branched chain hydrocarbon group is not particularly limited, and examples thereof include an i-propenyl group and a 3-butenyl group. The unsaturated-cyclic hydrocarbon group is not particularly limited, and examples thereof include a cyclopentenyl group and a cyclohexenyl group. The aromatic group is not particularly limited, and examples thereof include a phenyl group and a naphthalene group.

In addition, the derived group in R is not particularly limited, and examples thereof include functional groups in which at least one type of functional groups such as a carboxyl group, a hydroxy group, and an amino group is added or substituted with respect to the main chain or the side chain of the various hydrocarbon groups. In addition, the number of carbon atoms constituting the main chain of R is not particularly limited, and is preferably 20 or less and more preferably 10 or less. When the number of carbon atoms constituting the main chain of R is set to be within the above range, the molecular weight of the phosphorus oxoacid group can be set to be within an appropriate range, penetration into the fiber raw material is facilitated, and the yield of the fine fibrous celluloses can increase.

β^{b+} is a monovalent or higher cation composed of an organic substance or an inorganic substance. Examples of monovalent or higher cations composed of an organic substance include aliphatic ammonium and aromatic ammonium, and monovalent or higher cations composed of an inorganic substance are not particularly limited, and examples thereof include ions of alkali metals such as sodium, potassium, and lithium, cations of divalent metals such as calcium and magnesium, and hydrogen ions. These may be applied alone or two or more thereof may be used in combination. The monovalent or higher cation composed of an organic substance or an inorganic substance is not particularly limited, and is preferably a sodium or potassium ion because a fiber raw material containing β is unlikely to turn yellow when heated and is easy to use industrially. Here, β^{b+} may be an organic onium ion, and in this case, an organic ammonium ion is particularly preferable.

Examples of substituents derived from a carboxyl group include a carboxylic acid metal base, a carboxylic acid ionic group (—COO—), a carboxyalkyl group, and an alkylcarboxyl group. In the carboxyalkyl group or alkylcarboxyl group, the number of carbon atoms of the alkyl group is, for example, preferably 1 or more and 10 or less, more preferably 1 or more and 6 or less, and still more preferably 1 or more and 3 or less. Specific examples of alkyl groups include linear alkyl groups such as a methyl group, an ethyl group, an n-propyl group, and an n-butyl group, and branched chain alkyl groups such as an i-propyl group and a t-butyl group. Here, the substituent derived from a carboxyl group may be contained as a group in which carboxyl groups are condensed (for example, a carboxylic anhydride group) in the fine fibrous cellulose. A carboxyl group and a substituent derived from a carboxyl group are preferably introduced by a TEMPO oxidation treatment.

In addition, the sulfur oxoacid group (a sulfur oxoacid group or a substituent derived from a sulfur oxoacid group) is, for example, a substituent represented by the following Formula (2).

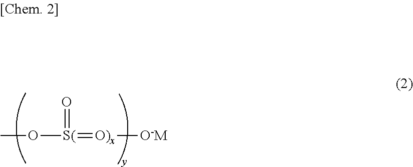

In the structural formula, y is a natural number, and x is 0 or 1. Here, when y is 2 or more, a plurality of x may be the same number or different numbers. In the structural formula, M is a monovalent or higher cation composed of an organic substance or an inorganic substance. Examples of monovalent or higher cations composed of an organic substance include aliphatic ammonium and aromatic ammonium, and examples of monovalent or higher cations composed of an inorganic substance include ions of alkali metals such as sodium, potassium, and lithium, cations of divalent metals such as calcium and magnesium, hydrogen ions, and ammonium ions, but the present invention is not particularly limited. These may be applied alone or two or more thereof may be used in combination. The monovalent or higher cation composed of an organic substance or an inorganic substance is not particularly limited, and is preferably an ammonium ion, a sodium ion, or a potassium ion so that it is easy to use industrially.

The amount of ionic substituents introduced into the fine fibrous cellulose is, for example, preferably 0.10 mmol/g or more, more preferably 0.20 mmol/g or more, still more preferably 0.50 mmol/g or more, and particularly preferably 1.00 mmol/g or more, per 1 g (mass) of the fine fibrous cellulose. In addition, the amount of ionic substituents introduced into the fine fibrous cellulose is, for example, preferably 5.20 mmol/g or less, more preferably 3.65 mmol/g or less, still more preferably 3.50 mmol/g or less, and particularly preferably 3.00 mmol/g or less, per 1 g (mass) of the fine fibrous cellulose. When the amount of ionic substituents introduced is set to be within the above range, it is possible to facilitate micronizing of the fiber raw material, and it is possible to improve the stability of the fine fibrous cellulose. Here, the denominator in the unit mmol/g indicates the mass of the fine fibrous cellulose when the counterion of the ionic substituent is a hydrogen ion (W).

The amount of ionic substituents introduced into the fine fibrous cellulose can be measured by, for example, a neutralization titration method. In measurement by the neutralization titration method, the change in the pH is determined while an alkali such as a sodium hydroxide aqueous solution is added to the obtained slurry containing fine fibrous celluloses, and thus the introduced amount is measured.

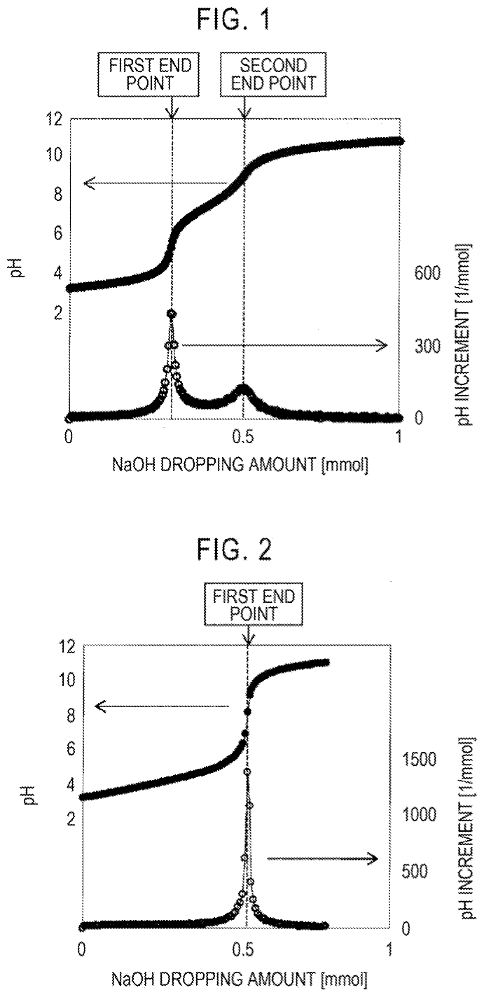

FIG. 1 is a graph showing the relationship between a NaOH dropping amount and a pH of a dispersion solution containing fine fibrous celluloses having a phosphorus oxoacid group as an ionic substituent. The amount of phosphorus oxoacid groups introduced into the fine fibrous cellulose is measured, for example, as follows.

First, the dispersion solution containing fine fibrous celluloses is treated with a strongly acidic cation exchange resin. Here, as necessary, before treatment with a strongly acidic cation exchange resin, the same defibration treatment as in a defibration treatment process to be described below may be performed on a measurement target.

Next, the change in the pH while a sodium hydroxide aqueous solution is added is observed, and a titration curve shown in the upper part in FIG. 1 is obtained. The titration curve shown in the upper part in FIG. 1 plots the pH measured with respect to the amount of an alkali added, and the titration curve shown in the lower part in FIG. 1 plots the pH increment (differential value) (1/mmol) with respect to the amount of an alkali added. In this neutralization titration, two points at which the increment (a differential value of the pH with respect to the amount of an alkali dropped) becomes a maximum are confirmed in the curve plotting the pH measured with respect to the amount of an alkali added. Among these, the maximum point of the increment obtained first when the alkali is added is called a first end point, and the maximum point of the increment obtained next is called a second end point. The amount of an alkali required from the titration start to the first end point is equal to the amount of the first dissociated acids of the fine fibrous cellulose contained in the dispersion solution used for titration, the amount of an alkali required from the first end point to the second end point is equal to the amount of the second dissociated acids of the fine fibrous cellulose contained in the slurry used for titration, and the amount of an alkali required from the titration start to the second end point is equal to the total amount of dissociated acids of the fine fibrous cellulose contained in the dispersion solution used for titration. Here, the value obtained by dividing the amount of an alkali required from the titration start to the first end point by the solid content (g) in the slurry to be titrated is the amount of phosphorus oxoacid groups introduced (mmol/g). Here, the amount of phosphorus oxoacid groups introduced (or the amount of phosphorus oxoacid groups) is simply an amount of the first dissociated acids.

Here, in FIG. 1, the region from the titration start to the first end point is called a first region, and the region from the first end point to the second end point is called a second region. For example, when the phosphorus oxoacid group is a phosphoric acid group and the phosphoric acid group causes condensation, the amount of weakly acidic groups (the amount of the second dissociated acids) in the phosphorus oxoacid group appears to decrease, and the amount of an alkali required for the second region is smaller than the amount of an alkali required for the first region. On the other hand, the amount of strongly acidic groups (the amount of the first dissociated acids) in the phosphorus oxoacid group corresponds to the amount of phosphorus atoms regardless of presence or absence of condensation. In addition, when the phosphorus oxoacid group is a phosphite group, since there is no weakly acidic group in the phosphorus oxoacid group, the amount of an alkali required for the second region is small, but the amount of an alkali required for the second region may become zero. In this case, on the titration curve, there is one point at which the pH increment is a maximum.

Here, the amount of phosphorus oxoacid groups introduced (mmol/g) indicates an amount of phosphorus oxoacid groups contained in the acid type fine fibrous cellulose (hereinafter referred to as the amount of phosphorus oxoacid groups (acid type)) because the denominator indicates the mass of the acid type fine fibrous cellulose. On the other hand, when the counterion of the phosphorus oxoacid group is replaced with an arbitrary cation C so that charges are equivalent, the denominator is converted to the mass of the fine fibrous cellulose when the cation C is a counterion, and thus the amount of phosphorus oxoacid groups contained in the fine fibrous cellulose in which the cation C is a counterion (hereinafter referred to as the amount of phosphorus oxoacid groups (C type)) can be determined. That is, the amount is calculated by the following computation formula.

Amount of phosphorus oxoacid groups (C type)=amount of phosphorus oxoacid groups (acid type)/{1−4W−1)×A/1,000}

A [mmol/g]: total amount of anions derived from phosphorus oxoacid groups contained in fine fibrous cellulose (total amount of dissociated acids of phosphorus oxoacid groups)

W: formula weight per valence of cation C (for example, Na is 23, and Al is 9)

FIG. 2 is a graph showing the relationship between a NaOH dropping amount and a pH of the dispersion solution containing fine fibrous celluloses having carboxyl groups as ionic substituents. The amount of carboxyl groups introduced into the fine fibrous cellulose is measured, for example, as follows.

First, the dispersion solution containing fine fibrous celluloses is treated with a strongly acidic cation exchange resin. Here, as necessary, before treatment with a strongly acidic cation exchange resin, the same defibration treatment as in a defibration treatment process to be described below may be performed on a measurement target.

Next, the change in the pH is observed while a sodium hydroxide aqueous solution is added, and a titration curve as shown in the upper part in FIG. 2 is obtained. The titration curve shown in the upper part in FIG. 2 plots the measured pH with respect to the amount of an alkali added, and the titration curve shown in the lower part in FIG. 2 plots a pH increment (differential value) (1/mmol) with respect to the amount of an alkali added. In this neutralization titration, one point at which the increment (the differential value of the pH with respect to the amount of an alkali dropped) becomes a maximum is confirmed in the curve plotting the measured pH with respect to the amount of an alkali added, and this maximum point is called a first end point. Here, the region from the titration start to the first end point in FIG. 2 is called a first region. The amount of an alkali required for the first region corresponds to the amount of carboxyl groups in the dispersion solution used for titration. Here, the amount of an alkali (mmol) required in the first region of the titration curve is divided by the solid content (g) in the dispersion solution containing fine fibrous celluloses to be titrated, and thus the amount of carboxyl groups introduced (mmol/g) is calculated.

Here, the amount of carboxyl groups introduced (mmol/g) indicates an amount of carboxyl groups contained in the acid type fine fibrous celluloses (hereinafter referred to as the amount of carboxyl groups (acid type)) because the denominator indicates the mass of acid type fine fibrous celluloses. On the other hand, when the counterion of the carboxyl group is replaced with an arbitrary cation C so that charges are equivalent, the denominator is converted to the mass of the fine fibrous cellulose when the cation C is a counterion, and thus the amount of carboxyl groups contained in the fine fibrous cellulose in which the cation C is a counterion (hereinafter referred to as the amount of carboxyl groups (C type)) can be determined. That is, the amount is calculated by the following computation formula.

Amount of carboxyl groups (C type)=amount of carboxyl groups (acid type)/{1+(W−1)×(amount of carboxyl groups (acid type))/1,000}

W: formula weight per valence of cation C (for example, Na is 23, and Al is 9)

In the measurement of the amount of ionic substituents according to the titration method, if the amount of one drop of the sodium hydroxide aqueous solution is too large or if the titration interval is too short, an inaccurate value such as an amount of ionic substituents that is smaller than the original amount may be obtained. As an appropriate dropping amount and titration interval, it is desirable to titrate, for example, 10 μL to 50 μL of a 0.1 N sodium hydroxide aqueous solution for 5 seconds to 30 seconds. In addition, in order to eliminate the influence of carbon dioxide dissolved in the dispersion solution containing fine fibrous celluloses, for example, from 15 minutes before the titration starts until the titration ends, it is desirable to perform measurement while blowing an inert gas such as nitrogen gas into a slurry.

In addition, the amount of sulfur oxoacid groups introduced into the fine fibrous cellulose can be calculated by freeze-drying the slurry containing fine fibrous celluloses and measuring the amount of sulfur in the crushed sample. Specifically, the slurry containing fine fibrous celluloses is freeze-dried and the crushed sample is pressurized, heated and decomposed with nitric acid in a closed container, and then diluted appropriately, and the amount of sulfur is measured through ICP-OES. The value calculated by performing division by the absolute dry mass of the test fine fibrous celluloses is used as an amount of sulfur oxoacid groups (unit: mmol/g) of the fine fibrous celluloses.

In one or more embodiments of the present invention, the fine fibrous cellulose is not particularly limited, for example, and it can be obtained by defibrating ionic-substituent-introduced fibers obtained by introducing an ionic substituent into a fiber raw material containing celluloses. In the ionic-substituent-introduced fibers, some hydroxy groups contained in cellulose molecules are substituted with ionic substituents or converted into ionic substituents.

<Fiber Raw Material>

Fine fibrous celluloses are produced from a fiber raw material containing celluloses. The fiber raw material containing celluloses is not particularly limited, and pulp is preferably used because it is easily available and inexpensive. Examples of pulp include wood pulp, non-wood pulp, and de-inked pulp. The wood pulp is not particularly limited, and examples thereof include chemical pulp such as leaf bleached kraft pulp (LBKP), needle bleached kraft pulp (NBKP), sulphite pulp (SP), dissolving pulp (DP), alkaline pulp (AP), unbleached kraft pulp (UKP) and oxygen bleached kraft pulp (OKP), semi-chemical pulp such as semi-chemical pulp (SCP) and chemi-ground wood pulp (CGP), and mechanical pulp such as ground wood pulp (GP) and thermomechanical pulp (TMP, BCTMP). The non-wood pulp is not particularly limited, and examples thereof include cotton pulp such as cotton linter and cotton linter, and non-wood pulp such as hemp, straw and bagasse. The de-inked pulp is not particularly limited, and examples thereof include de-inked pulp using waste paper as a raw material. These pulps may be used alone or two or more thereof may be used in combination. Among the above pulps, for example, wood pulp and de-inked pulp are preferable because they are easily available. In addition, among wood pulp, in order to increase the cellulose ratio and increase the yield of the fine fibrous cellulose during a defibration treatment and in order to obtain fine fibrous celluloses of long fibers with weak decomposition of cellulose in pulp and a large axial ratio, for example, chemical pulp is more preferable, and kraft pulp and sulphite pulp are more preferable. Here, the viscosity tends to increase when fine fibrous celluloses of long fibers having a large axial ratio are used.

As the fiber raw material containing celluloses, for example, cellulose contained in ascidians and bacterial cellulose produced from acetic acid bacteria can be used. In addition, in place of the fiber raw material containing celluloses, fibers formed of a linear nitrogen-containing polysaccharide polymer such as chitin or chitosan can also be used.

<Phosphorus Oxoacid Group Introduction Process>

A process of producing fine fibrous celluloses preferably includes an ionic substituent introduction process, and examples of ionic substituent introduction processes include a phosphorus oxoacid group introduction process. The phosphorus oxoacid group introduction process is a process in which at least one compound (hereinafter referred to as a “compound A”) selected from among compounds that can introduce a phosphorus oxoacid group is made to act on a fiber raw material containing celluloses, hydroxy groups of the fiber raw material containing celluloses are reacted with a compound that can introduce a phosphorus oxoacid group, some hydroxy groups contained in the fiber raw material containing celluloses are substituted with ionic substituents, and thus phosphorus oxoacid group-introduced fibers are obtained.

In the phosphorus oxoacid group introduction process, the reaction between the fiber raw material containing celluloses and the compound A may be performed in the presence of at least one selected from among urea and derivatives thereof (hereinafter referred to as a “compound B”). On the other hand, the reaction between the fiber raw material containing celluloses and the compound A may be performed in the absence of the compound B.

As an example of a method of allowing the compound A to act on the fiber raw material in the co-presence of the compound B, a method of mixing the compound A and the compound B into a dry or wet slurry-like fiber raw material may be exemplified. Particularly, in order to improve the reaction uniformity, it is preferable to use a dry or wet fiber raw material, and a dry fiber raw material is particularly preferably used. The form of the fiber raw material is not particularly limited, and for example, a cotton form or a thin sheet form is preferable. The compound A and the compound B in the form of powder, in the form of a solution dissolved in a solvent or in a form in which the compound is melted by heating to a melting point or more may be added to the fiber raw material. Particularly, in order to improve the reaction uniformity, it is preferable to add the compound in the form of a solution dissolved in a solvent, and particularly, in the form of an aqueous solution. In addition, the compound A and the compound B may be added to the fiber raw material at the same time, or may be added separately, or may be added as a mixture. A method of adding the compound A and the compound B is not particularly limited, and when the compound A and the compound B are in the form of a solution, the fiber raw material may be immersed in a solution, the solution may be absorbed and then taken out, and the solution may be added dropwise to the fiber raw material. In addition, required amounts of the compound A and the compound B may be added to the fiber raw material, or excess amounts of the compound A and the compound B may be added to the fiber raw material, and the excess compound A and compound B may then be removed by squeezing or filtering.

The compound A used in the present embodiment may be any compound which has phosphorus atoms and can form an ester bond with cellulose, and examples thereof include phosphoric acid or salts thereof, phosphites or salts thereof, dehydration-condensed phosphoric acid or salts thereof, and phosphoric acid anhydride (diphosphorus pentoxide), but the present invention is not particularly limited. As the phosphoric acid, those having various purities can be used, and for example, 100% phosphoric acid (orthophosphoric acid) or 85% phosphoric acid can be used. Examples of phosphites include 99% phosphites (phosphonic acid). The dehydration-condensed phosphoric acid is an acid in which two or more molecules of phosphoric acid are condensed by a dehydration reaction, and examples thereof include pyrophosphoric acid and polyphosphoric acid. Examples of phosphates, phosphites, and dehydration-condensed phosphates include phosphoric acid, phosphites or dehydration-condensed phosphoric acid lithium salts, sodium salts, potassium salts, and ammonium salts, and these can have various degrees of neutralization. Among these, in consideration of high phosphoric acid group introduction efficiency, ease of further improvement of the defibration efficiency in a defibration process to be described below, low cost, and ease of industrial application, one or more selected from the group consisting of phosphoric acid, a sodium salt of phosphoric acid, a potassium salt of phosphoric acid, an ammonium salt of phosphoric acid, phosphites, a sodium salt of phosphites, a potassium salt of phosphites, and an ammonium salt of phosphites are preferable, and one or more selected from the group consisting of phosphoric acid, sodium dihydrogen phosphate, disodium hydrogen phosphate, ammonium dihydrogen phosphate, phosphites, and sodium phosphite are more preferable.

The amount of the compound A added to the fiber raw material is not particularly limited, and for example, when the amount of the compound A added is converted to the amount of phosphorus atoms, the amount of phosphorus atoms added to the fiber raw material (absolute dry mass) is preferably 0.5 mass % or more and 100 mass % or less, more preferably 1 mass % or more and 50 mass % or less, and still more preferably 2 mass % or more and 30 mass % or less. When the amount of phosphorus atoms added to the fiber raw material is set to be within the above range, it is possible to further improve the yield of the fine fibrous cellulose. On the other hand, when the amount of phosphorus atoms added to the fiber raw material is set to the upper limit value or less, the effect of improving the yield and cost can be balanced.

The compound B used in the present embodiment is at least one selected from among urea and derivatives thereof as described above. Examples of the compound B include urea, biuret, 1-phenylurea, 1-benzylurea, 1-methyl urea, and 1-ethylurea. In addition, in order to further improve the reaction uniformity, it is preferable to use an aqueous solution in which both the compound A and the compound B are dissolved.

The amount of the compound B added to the fiber raw material (absolute dry mass) is not particularly limited, and is, for example, preferably 1 mass % or more and 500 mass % or less, more preferably 10 mass % or more and 400 mass % or less, and still more preferably 100 mass % or more and 350 mass % or less.

In the reaction between the fiber raw material containing celluloses and the compound A, in addition to the compound B, for example, amides or amines may be contained in the reaction system. Examples of amides include formamide, dimethyl formamide, acetamide, and dimethylacetamide. Examples of amines include methylamine, ethylamine, trimethylamine, triethylamine, monoethanolamine, diethanolamine, triethanolamine, pyridine, ethylenediamine, and hexamethylenediamine. Among these, it is known that trimethylamine acts as a particularly favorable reaction catalyst.

In the phosphorus oxoacid group introduction process, it is preferable to add or mix the compound A or the like to the fiber raw material and then heat the fiber raw material. As the heat treatment temperature, it is preferable to select a temperature at which the phosphorus oxoacid group can be efficiently introduced while restricting fiber thermal decomposition and a hydrolysis reaction. Specifically, the temperature is preferably 50° C. or higher and 300° C. or lower, more preferably 100° C. or higher and 250° C. or lower, and still more preferably 130° C. or higher and 200° C. or lower. In addition, a device having various heating mediums can be used for the heat treatment. For example, a stirring and drying device, a rotary drying device, a disk drying device, a roll type heating device, a plate type heating device, a fluidized bed drying device, a band type drying device, a filtering drying device, a vibration flow drying device, an airflow drying device, a vacuum drying device, an infrared heating device, a far infrared heating device, a microwave heating device, and a high frequency drying device can be used.

In the heat treatment according to the present embodiment, for example, a method of adding the compound A to a thin sheet-like fiber raw material by a method such as impregnation and then heating, or a method of heating a fiber raw material and the compound A while kneading or stirring with a kneader or the like can be used. Thereby, it is possible to reduce the uneven concentration of the compound A in the fiber raw material and more uniformly introduce a phosphorus oxoacid group to the surface of cellulose fibers contained in the fiber raw material. This is thought to be caused by the fact that, when water molecules move to the surface of the fiber raw material due to drying, the dissolved compound A is attracted to water molecules due to surface tension, and similarly, moving to the surface of the fiber raw material (that is, causing the uneven concentration of the compound A) can be restricted.

In addition, the heating device used for the heat treatment is preferably, for example, a device that can constantly discharge water retained in the slurry, and water generated according to a dehydration condensation (phosphate esterification) reaction between the compound A and hydroxy groups contained in the cellulose or the like in the fiber raw material to the outside of a device system. Examples of such heating devices include a ventilation type oven. When water in the device system is constantly discharged, it is possible to restrict a hydrolysis reaction of the phosphate ester bond, which is a reverse reaction of phosphate esterification, and also restrict acid hydrolysis at the sugar chain in the fiber. Therefore, it is possible to obtain fine fibrous celluloses having a high axial ratio.

The heat treatment time is, for example, preferably 1 second or more and 300 minutes or less, more preferably 1 second or more and 1,000 seconds or less, and still more preferably 10 seconds or more and 800 seconds or less after water is substantially removed from the fiber raw material. In the present embodiment, when the heating temperature and the heating time are set to be within appropriate ranges, the amount of phosphorus oxoacid groups introduced can be set to be within a preferable range.

The phosphorus oxoacid group introduction process may be performed at least once, but can be repeated twice or more. When the phosphorus oxoacid group introduction process is performed twice or more, a greater amount of phosphorus oxoacid groups can be introduced into the fiber raw material. In the present embodiment, as an example of a preferable embodiment, a case in which the phosphorus oxoacid group introduction process is performed twice may be exemplified.

The amount of phosphorus oxoacid groups introduced into the fiber raw material is, for example, preferably 0.10 mmol/g or more, more preferably 0.20 mmol/g or more, still more preferably 0.50 mmol/g or more, yet more preferably 1.00 mmol/g or more, and particularly preferably 1.20 mmol/g or more, per 1 g (mass) of the fine fibrous cellulose. In addition, the amount of phosphorus oxoacid groups introduced into the fiber raw material is, for example, preferably 5.20 mmol/g or less, more preferably 3.65 mmol/g or less, and still more preferably 3.00 mmol/g or less, per 1 g (mass) of the fine fibrous cellulose. When the amount of phosphorus oxoacid groups introduced is set to be within the above range, it is possible to facilitate micronizing of the fiber raw material and it is possible to improve the stability of the fine fibrous cellulose.

<Carboxyl Group Introduction Process>

The fine fibrous cellulose production process may include, for example, a carboxyl group introduction process, as the ionic substituent introduction process. The carboxyl group introduction process may be performed by treating a fiber raw material containing celluloses according to ozone oxidation or oxidation by a Fenton method, an oxidation treatment such as a TEMPO oxidation treatment, a compound having a group derived from a carboxylic acid or derivatives thereof, or treating with an acid anhydride of a compound having a group derived from a carboxylic acid or derivatives thereof, and is preferably performed according to a TEMPO oxidation treatment.

The compound having a group derived from a carboxylic acid is not particularly limited, and examples thereof include dicarboxylic acid compounds such as maleic acid, succinic acid, phthalic acid, fumaric acid, glutaric acid, adipic acid, and itaconic acid, and tricarboxylic acid compounds such as citric acid and aconitic acid. In addition, the derivative of the compound having a group derived from a carboxylic acid is not particularly limited, and examples thereof include an imidized product of an acid anhydride of a compound having a carboxyl group and derivatives of an acid anhydride of a compound having a carboxyl group. The imidized product of the acid anhydride of the compound having a carboxyl group is not particularly limited, and examples thereof include imidized products of dicarboxylic acid compounds such as maleimide, succinimide, and phthalate imide. In the treatment with these compounds, hydroxy groups of cellulose molecules and the compound having a group derived from a carboxylic acid or the like undergo a dehydration reaction to form a polar group (—COO—).

The acid anhydride of the compound having a group derived from a carboxylic acid is not particularly limited, and examples thereof include acid anhydrides of dicarboxylic acid compounds such as maleic anhydride, succinic anhydride, phthalic anhydride, glutaric anhydride, adipic acid anhydride, and itaconic acid anhydride. In addition, the derivative of the acid anhydride of the compound having a group derived from a carboxylic acid is not particularly limited, and examples thereof include those in which at least some hydrogen atoms of an acid anhydride of a compound having a carboxyl group such as didimethylmaleic anhydride, diethylmaleic anhydride, and diphenylmaleic anhydride are substituted with a substituent such as an alkyl group and a phenyl group.

In the carboxyl group introduction process, when the TEMPO oxidation treatment is performed, for example, it is preferable to perform this treatment under a condition of a pH of 6 or more and 8 or less. Such a treatment is also called a neutral TEMPO oxidation treatment. The neutral TEMPO oxidation treatment can be performed by, for example, adding pulp as a fiber raw material, a nitroxy radical such as TEMPO (2,2,6,6-tetramethylpiperidin-1-oxyl) as a catalyst, and sodium hypochlorite as a sacrificial reagent to a sodium phosphate buffer solution (pH=6.8). In addition, by allowing sodium chlorite to coexist, an aldehyde generated in an oxidation procedure can be efficiently oxidized to the carboxyl group. In addition, for the TEMPO oxidation treatment, this treatment may be performed under a condition of a pH of 10 or more and 11 or less. Such a treatment is also called an alkaline TEMPO oxidation treatment. The alkaline TEMPO oxidation treatment can be performed by, for example, adding a nitroxy radical such as TEMPO as a catalyst, sodium bromide as a cocatalyst, and sodium hypochlorite as an oxidant to pulp as a fiber raw material. According to the TEMPO oxidation treatment or the alkaline TEMPO oxidation treatment, some hydroxy groups contained in cellulose molecules are converted into carboxyl groups.

The amount of carboxyl groups introduced into the fiber raw material varies depending on the type of the substituent, but, for example, when carboxyl groups are introduced according to the TEMPO oxidation, the amount is preferably 0.10 mmol/g or more, more preferably 0.20 mmol/g or more, still more preferably 0.50 mmol/g or more, yet more preferably 0.90 mmol/g or more, and particularly preferably 1.40 mmol/g or more, per 1 g (mass) of the fine fibrous cellulose. In addition, the amount is preferably 2.50 mmol/g or less, more preferably 2.20 mmol/g or less, and still more preferably 2.00 mmol/g or less, per 1 g (mass) of the fine fibrous cellulose. In addition, when the substituent is a carboxymethyl group, the amount may be 5.8 mmol/g or less, per 1 g (mass) of the fine fibrous cellulose.

<Sulfur Oxoacid Group Introduction Process>

The fine fibrous cellulose production process may include, for example, a sulfur oxoacid group introduction process, as the ionic substituent introduction process. In the sulfur oxoacid group introduction process, cellulose fibers (sulfur oxoacid group-introduced fibers) having a sulfur oxoacid group can be obtained by reacting hydroxy groups contained in the fiber raw material containing celluloses with sulfur oxoacid.

In the sulfur oxoacid group introduction process, in place of the compound A in the above <Phosphorus oxoacid group introduction process>, at least one compound (hereinafter referred to as a compound C) selected from among compounds that can introduce a sulfur oxoacid group by reacting with a hydroxy group contained in the fiber raw material containing celluloses is used. The compound C may be any compound which has sulfur atoms and can form an ester bond with cellulose, and examples thereof include sulfuric acid (phosphonic acid) or salts thereof, sulfurous acid or salts thereof, and sulfuric acid amide, but the present invention is not particularly limited. As the sulfuric acid (phosphonic acid), those having various purities can be used, for example, 96% sulfuric acid (concentrated sulfuric acid) can be used. Examples of sulfurous acid include 5% sulfurous acid water. Examples of sulfates or sulfites include sulfuric acid or sulfurous acid lithium salts, sodium salts, potassium salts, and ammonium salts, and these can have various degrees of neutralization. As the sulfuric acid amide, sulfamic acid or the like can be used. In the sulfur oxoacid group introduction process, it is preferable to use the compound B in the above <Phosphorus oxoacid group introduction process> in the same manner.

In the sulfur oxoacid group introduction process, it is preferable to mix the cellulose raw material with an aqueous solution containing sulfur oxoacid, and urea and/or urea derivatives and then heat the cellulose raw material. As the heat treatment temperature, it is preferable to select a temperature at which the sulfur oxoacid group can be efficiently introduced while restricting fiber thermal decomposition and a hydrolysis reaction. The heat treatment temperature is preferably 100° C. or higher, more preferably 120° C. or higher, and still more preferably 150° C. or higher. In addition, the heat treatment temperature is preferably 300° C. or lower, more preferably 250° C. or lower, and still more preferably 200° C. or lower.

In the heat treatment process, it is preferable to heat until water is substantially eliminated. Therefore, the heat treatment time varies depending on the amount of water contained in the cellulose raw material, and the amount of the aqueous solution containing sulfur oxoacid, and urea and/or urea derivative added, but is preferably, for example, 10 seconds or more and 10,000 seconds or less. A device having various heating mediums can be used for the heat treatment, and for example, a hot air drying device, a stirring and drying device, a rotary drying device, a disk drying device, a roll type heating device, a plate type heating device, a fluidized bed drying device, a band type drying device, a filtering and drying device, a vibration flow drying device, an airflow drying device, a vacuum drying device, an infrared heating device, a far infrared heating device, a microwave heating device, and a high frequency drying device can be used.

The amount of sulfur oxoacid groups introduced into the fiber raw material is preferably 0.05 mmol/g or more, more preferably 0.10 mmol/g or more, still more preferably 0.20 mmol/g or more, yet more preferably 0.50 mmol/g or more, and particularly preferably 0.90 mmol/g or more. In addition, the amount of sulfur oxoacid groups introduced into the fiber raw material is preferably 5.00 mmol/g or less and more preferably 3.00 mmol/g or less. When the amount of sulfur oxoacid groups introduced is set to be within the above range, it is possible to facilitate micronizing of the fiber raw material, and it is possible to improve the stability of the fine fibrous cellulose.

<Washing Process>

In the method of producing fine fibrous celluloses according to the present embodiment, as necessary, a washing process can be performed on the ionic substituent-introduced fibers. The washing process is performed by washing the ionic substituent-introduced fibers with, for example, water or an organic solvent. In addition, the washing process may be performed after processes to be described below, and the number of washing performed in each washing process is not particularly limited.

<Alkaline Treatment Process>

When fine fibrous celluloses are produced, between the ionic substituent introduction process and the defibration treatment process to be described below, an alkaline treatment may be performed on the ionic substituent-introduced fibers. The alkaline treatment method is not particularly limited, and examples thereof include a method of immersing ionic substituent-introduced fibers in an alkaline solution.

The alkaline compound contained in the alkaline solution is not particularly limited, and may be an inorganic alkaline compound or an organic alkaline compound. In the present embodiment, it is preferable to use, for example, sodium hydroxide or potassium hydroxide as an alkaline compound, because it has high versatility. In addition, the solvent contained in an alkaline solution may be either water or an organic solvent. Among these, the solvent contained in the alkaline solution is preferably a polar solvent containing water or a polar organic solvent exemplified by an alcohol, and more preferably an aqueous solvent containing at least water. The alkaline solution is preferably, for example, a sodium hydroxide aqueous solution or a potassium hydroxide aqueous solution, because it has high versatility.

The temperature of the alkaline solution in the alkaline treatment process is not particularly limited, and is, for example, preferably 5° C. or higher and 80° C. or lower, and more preferably 10° C. or higher and 60° C. or lower. The time of immersing the ionic substituent-introduced fibers in an alkaline solution in the alkaline treatment process is not particularly limited, and is, for example, preferably 5 minutes or more and 30 minutes or less and more preferably 10 minutes or more and 20 minutes or less. The amount of the alkaline solution used in the alkaline treatment is not particularly limited, and is, for example, preferably 100 mass % or more and 100,000 mass % or less and more preferably 1,000 mass % or more and 10,000 mass % or less, with respect to the absolute dry mass of the ionic substituent-introduced fibers.

In order to reduce the amount of the alkaline solution used in the alkaline treatment process, the ionic substituent-introduced fibers may be washed with water or an organic solvent after the ionic substituent introduction process and before the alkaline treatment process. After the alkaline treatment process and before the defibration treatment process, in order to improve handling properties, it is preferable to wash the ionic substituent-introduced fibers subjected to the alkaline treatment with water or an organic solvent.

<Acid Treatment Process>

When fine fibrous celluloses are produced, between the ionic substituent introduction process and the defibration treatment process to be described below, an acid treatment may be performed on the fiber raw material. For example, the ionic substituent introduction process, the acid treatment, the alkaline treatment and the defibration treatment may be performed in that order.

The acid treatment method is not particularly limited, and examples thereof include a method of immersing the fiber raw material in an acidic liquid containing an acid. The concentration of the acidic liquid used is not particularly limited, and is, for example, preferably 10 mass % or less, and more preferably 5 mass % or less. In addition, the pH of the acidic liquid used is not particularly limited, and is, for example, preferably 0 or more and 4 or less and more preferably 1 or more and 3 or less. As the acid contained in an acidic liquid, for example, an inorganic acid, a sulfonic acid, and a carboxylic acid can be used. Examples of inorganic acids include sulfuric acid, nitric acid, hydrochloric acid, hydrobromic acid, hydriodic acid, hypochlorous acid, chlorous acid, chloric acid, perchloric acid, phosphoric acid, and boric acid. Examples of sulfonic acids include methane sulfonic acid, ethane sulfonic acid, benzene sulfonic acid, p-toluene sulfonic acid, and trifluoromethane sulfonic acid. Examples of carboxylic acids include formic acid, acetic acid, citric acid, gluconic acid, lactic acid, oxalic acid, and tartaric acid. Among these, hydrochloric acid or sulfuric acid is particularly preferably used.

The temperature of the acid solution in the acid treatment is not particularly limited, and is, for example, preferably 5° C. or higher and 100° C. or lower and more preferably 20° C. or higher and 90° C. or lower. The time of immersion in an acid solution in the acid treatment is not particularly limited, and is, for example, preferably 5 minutes or more and 120 minutes or less and more preferably 10 minutes or more and 60 minutes or less. The amount of the acid solution used in the acid treatment is not particularly limited, and is, for example, preferably 100 mass % or more and 100,000 mass % or less and more preferably 1,000 mass % or more and 10,000 mass % or less, with respect to the absolute dry mass of the fiber raw material.

<Defibration Treatment Process>

Fine fibrous celluloses are obtained by defibrating the ionic substituent-introduced fibers in the defibration treatment process. In the defibration treatment process, for example, a defibration treatment device can be used. The defibration treatment device is not particularly limited, and for example, a high-speed defibrating machine, a grinder (stone mill type grinder), a high-pressure homogenizer, an ultra high-pressure homogenizer, a high pressure collision type grinder, a ball mill, a bead mill, a disc type refiner, a conical refiner, a twin-screw kneader, a vibration mill, a homo mixer under high-speed rotation, an ultrasonic disperser, or a beater can be used. Among the defibration treatment devices, it is more preferable to use a high-speed defibrating machine, a high-pressure homogenizer, and an ultra high-pressure homogenizer, which are influenced less by crushing media and have a low risk of contamination.

In the defibration treatment process, for example, it is preferable to dilute ionic substituent-introduced fibers with a dispersion medium to form a slurry. As the dispersion medium, one or two or more selected from among water and organic solvents such as a polar organic solvent can be used. The polar organic solvent is not particularly limited, and preferable examples thereof include alcohols, multivalent alcohols, ketones, ethers, esters, and aprotonic polar solvents. Examples of alcohols include methanol, ethanol, n-propanol, isopropanol, n-butanol, and t-butyl alcohol. Examples of multivalent alcohols include ethylene glycol, propylene glycol, and glycerin. Examples of ketones include acetone, and methyl ethyl ketone (MEK). Examples of ethers include diethyl ether, tetrahydrofuran (THF), ethylene glycol monomethyl ether, ethylene glycol monoethyl ether, ethylene glycol mono n-butyl ether, and propylene glycol monomethyl ether. Examples of esters include ethyl acetate and butyl acetate. Examples of aprotonic polar solvents include dimethyl sulfoxide (DMSO), dimethylformamide (DMF), dimethylacetamide (DMAc), and N-methyl-2-pyrrolidinone (NMP).

The solid content concentration of the ionic substituent-introduced fibers during the defibration treatment can be appropriately set. In addition, a slurry obtained by dispersing the ionic substituent-introduced fibers in a dispersion medium may contain, for example, a solid content other than the ionic substituent-introduced fibers such as urea having a hydrogen-bonding property.

In one or more embodiments of the present invention, a hydrogel is preferably formed of crosslinked hydrophilic polymers. In the hydrogel, it is preferable that fine fibrous celluloses be dispersed and embedded in a mesh structure of hydrophilic polymers crosslinked to each other. In one or more embodiments of the present invention, unless otherwise specified, “hydrophilic polymers” may be crosslinked hydrophilic polymers or uncrosslinked hydrophilic polymers, and “crosslinked hydrophilic polymers” means only crosslinked hydrophilic polymers. The hydrophilic polymers are preferably polymers that can be crosslinked by radiation emission to form a gel. Examples of hydrophilic polymers that are crosslinked by radiation emission to form a gel (hereinafter referred to as a radiation-crosslinkable hydrophilic polymer) include polyvinyl alcohol, polyvinylpyrrolidone, carboxymethyl cellulose, carboxymethyl cellulosesodium, polyacrylamide, polyacryloyl morpholine, water-soluble polyvinyl acetal, poly-N-vinylacetamide, hydroxyethyl cellulose, hydroxypropyl cellulose, methyl cellulose, hydroxypropyl methyl cellulose, hydroxyethyl methyl cellulose, gelatin, and casein, and derivatives thereof. Examples of derivatives of these radiation-crosslinkable hydrophilic polymers include derivatives in which various monomers are copolymerized or graft-polymerized, derivatives obtained by, for example, etherifying, esterifying, amidating, or acetalizing hydroxy groups, amino groups, amide groups, or carboxyl groups of the resin, and derivatives partially crosslinked with a crosslinking agent.

In addition, in order to improve absorbability of an exudate, adhesion to a living body and the like, other hydrophilic polymers may be contained in addition to the radiation-crosslinkable hydrophilic polymers. As the other hydrophilic polymers, for example, synthetic hydrophilic polymers, semi-synthetic hydrophilic polymers, or various natural hydrophilic polymers can be used.

In one or more embodiments of the present invention, the synthetic hydrophilic polymers are not particularly limited, and for example, vinyl hydrophilic polymers, acrylic hydrophilic polymers, polyethyleneimine, and polyethylene oxide can be used. Examples of vinyl hydrophilic polymers include polyvinyl methyl ether and carboxyvinyl polymers. Examples of acrylic hydrophilic polymers include sodium polyacrylate.

In one or more embodiments of the present invention, the semi-synthetic hydrophilic polymers are not particularly limited, and for example, starch-based polymers, cellulose-based polymers, and alginic acid-based polymers can be used. Examples of starch-based polymers include carboxymethyl starch and methyl hydroxypropyl starch. Examples of cellulose-based polymers include ethyl cellulose and cellulose sulfate sodium salts. Examples of alginic acid-based polymers include alginic acid sodium and propylene glycol alginate.

In one or more embodiments of the present invention, the natural hydrophilic polymer compound is not particularly limited, for example, plant-based polymers, microorganism-based polymers, and animal-based polymers can be used. Specific examples of plant-based polymers include gum arabic, tragacanth gum, galactan, guar gum, carob gum, karaya gum, carrageenan, pectin, agar, and starch (for example, rice, corn, potatoes, and wheat starch). Specific examples of microorganism-based polymers include xanthan gum, dextrin, dextran, succinoglucan, and pullulan. Specific examples of animal-based polymers include albumin.

The above hydrophilic polymers may be used alone or two or more thereof may be used in combination, and a copolymer having two or more types of frameworks or a mixture containing two or more types thereof may be used.

In one or more embodiments of the present invention, in consideration of ease of dispersion of fine fibrous celluloses, ease of improvement in the strength and water retention when radiation is emitted in the presence of fine fibrous celluloses to form a hydrogel, and ease of achievement of both adhesiveness and peelability with respect to skin, hydrophilic polymers more preferably contain one or more selected from the group consisting of polyvinyl alcohol (hereinafter simply referred to as “PVA”), polyvinylpyrrolidone and carboxymethyl cellulosesodium, and still more preferably contain polyvinyl alcohol.

In one or more embodiments of the present invention, the polyvinyl alcohol is not particularly limited, and the average degree of polymerization measured according to JIS K 6726: 1994 is, for example, preferably 300 or more and 5,000 or less and more preferably 1,000 or more and 4,000 or less. In addition, the degree of saponification of PVA (mol % of vinyl alcohol units of PVA) measured according to JIS K 6726: 1994 is not particularly limited, and is, for example, preferably 60 mol % or more and 100 mol % or less, more preferably 70 mol % or more and 100 mol % or less, and still more preferably 80 mol % or more and 100 mol % or less. When the average degree of polymerization and the degree of saponification of PVA are set to be within the above range, it is possible to easily form a hydrogel having favorable water absorption and ease of water retention.

The content of the fine fibrous cellulose in the hydrogel is not particularly limited, and is preferably 0.2 mass % or more and 1.8 mass % or less, more preferably 0.6 mass % or more and 1.8 mass % or less, and particularly preferably 1.2 mass % or more and 1.8 mass % or less. When the content of the fine fibrous cellulose is within the above range, it is easy to improve the water retention and strength of the hydrogel, and it is easy to achieve both the adhesiveness and peelability with respect to skin.

The radiation that causes mutual crosslinking of radiation-crosslinkable hydrophilic polymers is not particularly limited, and examples thereof include α-rays, β-rays, γ-rays, X-rays, electron beams, visible light, ultraviolet rays, and infrared rays. Among these radiations, γ-rays, X-rays, electron beams, visible light, or ultraviolet rays are preferable, and γ-rays or electron beams are more preferable, and electron beams are still more preferable because it is easy to control the dose, a sterilization treatment is also performed at the same time, and the productivity is favorable.

Since the hydrophilic polymers used in the hydrogel are crosslinked to each other by the radiation emission, a gel can be formed without using a separate crosslinking agent. Therefore, when a hydrogel containing no crosslinking agent is use, the safety can be improved.

The hydrogel can contain water due to a mesh structure of the hydrophilic polymers crosslinked to each other. The content of water in the hydrogel is not particularly limited, and is preferably 80 mass % or more and 98 mass % or less and more preferably 82 mass % or more and 96 mass % or less.

In one or more embodiments of the present invention, the hydrogel may contain, as necessary, an antibacterial agent, a preservative, an antioxidant, an antifoaming agent, a stabilizer, a surfactant, a plasticizer, a tackifier, a viscosity adjusting agent, a colorant, a medical component and the like.

In one or more embodiments of the present invention, the hydrogel can be prepared using a hydrogel composition obtained by mixing hydrophilic polymers and fine fibrous celluloses. Specifically, a hydrogel can be formed by emitting radiation to a hydrogel composition and crosslinking hydrophilic polymers. In the hydrogel obtained in this manner, fine fibrous celluloses are dispersed and embedded in a mesh structure of the crosslinked hydrophilic polymers.

In one or more embodiments of the present invention, the hydrogel composition can be prepared by adding and dissolving hydrophilic polymers to and in water and then adding and dispersing fine fibrous celluloses to and in the obtained hydrophilic polymer aqueous solution. In order to improve the dispersibility of fine fibrous celluloses, it is preferable to prepare the sample by mixing a hydrophilic polymer aqueous solution and fine fibrous cellulose water dispersion solution. In addition, in the mixing process, as necessary, stirring may be performed under conditions such as heating and depressurization.

The hydrophilic polymer aqueous solution is not particularly limited, and for example, in consideration of productivity, it is preferable to contain 10 mass % or more and 90 mass % or less of hydrophilic polymers, and more preferable to contain 10 mass % or more and 50 mass % or less of hydrophilic polymers.

The fine fibrous cellulose water dispersion solution is not particularly limited, and for example, in consideration of handling properties, it is preferable to contain 0.1 mass % or more and 15 mass % or less and more preferable to contain 1.0 mass % or more and 3.0 mass % or less of fine fibrous celluloses.

In one or more embodiments of the present invention, the hydrogel composition may contain, as necessary, an antibacterial agent, a preservative, an antioxidant, an antifoaming agent, a stabilizer, a surfactant, a plasticizer, a tackifier, a viscosity adjusting agent, a colorant, a medical component and the like.

The amount of radiation emission when the hydrogel is formed is not particularly limited as long as a crosslink reaction occurs. For example, when a crosslink reaction is caused by emission of γ-rays, X-rays, electron beams or the like, the cumulative radiation dose can be generally in a range of 0.1 kGy or more and 1,000 kGy or less, and preferably 1 kGy or more and 100 kGy or less. When the cumulative radiation dose is set to be within the above range, it is possible to control a crosslink reaction to the extent that it has an appropriate cohesion (gel strength) and water absorption. Therefore, it is preferable to appropriately set the cumulative radiation dose of radiation according to the type of the raw material used such as hydrophilic polymers so that a hydrogel having favorable cohesion and water absorption can be formed.

When electron beams are emitted, the electron beam radiation device is not particularly limited, and a curtain method, a scanning method or a double scanning method may be used. The acceleration voltage of electron beams according to this electron beam emission is not particularly limited, and may be, for example, in a range of 100 kV or more and 1,000 kV or less. In addition, the cumulative radiation dose of electron beams is not particularly limited, and may be, for example, in a range of 5 kGy or more and 100 kGy or less.

When emitting radiation such as electron beams, for example, it is preferable to place, accommodate, or fill the hydrogel composition on, in, or into a sheet-like substrate, a package, a molding die or the like so that radiation such as electron beams is easily emitted to the hydrogel composition or the hydrogel composition is easily cured. Electron beams are preferably emitted when the hydrogel composition is spread out so that the thickness is 0.05 mm or more and 5 mm or less. After electron beams are emitted, the hydrogel may be washed with water.

In one or more embodiments of the present invention, the wound covering material may include a support layer laminated on the side opposite to the skin side of the hydrogel (layer). For the support layer, various flexible and moisture-permeable non-woven fabrics and films can be used, but a polyurethane film or polyurethane foam is more preferable in order to maintain a moist environment suitable for wound healing and provide cushioning properties and protection properties to the wound part. The support layer is responsible for fixing the hydrogel to the wound part, protecting the wound part from external stimuli, and maintaining the covering material in a wet state suitable for wound healing.

The wound covering material may further include an intermediate layer for better anchoring and integrating the hydrogel (layer) and the support layer. In particular, when a hydrophobic adhesive, for example, an acrylic adhesive, is laminated between the support layer and the intermediate layer, the hydrophobic adhesive and the hydrophilic hydrogel can be integrated.

As the material used for the intermediate layer, various non-woven fabrics or films can be used, and PVA non-woven fabrics or PVA films are preferable in consideration of favorable compatibility with the hydrogel layer and transparency, and PVA non-woven fabrics are preferable in consideration of flexibility.

The present invention is not particularly limited, and preferably includes the following aspects.

[1] A wound covering material containing at least a hydrogel,

wherein the hydrogel contains fine fibrous celluloses having ionic substituents.

[2] The wound covering material according to [1],

wherein the ionic substituent is one or more anionic groups selected from the group consisting of a phosphorus oxoacid group, a substituent derived from a phosphorus oxoacid group, a carboxyl group, a substituent derived from a carboxyl group, a sulfur oxoacid group, and a substituent derived from a sulfur oxoacid group.

[3] The wound covering material according to [1] or [2],

wherein the fine fibrous celluloses have an average fiber width of 1,000 nm or less.

[4] The wound covering material according to any one of [1] to [3],

wherein the hydrogel is formed of crosslinked hydrophilic polymers.

[5] The wound covering material according to [4],

wherein the hydrophilic polymer is one or more selected from the group consisting of polyvinyl alcohol, polyvinylpyrrolidone, and carboxymethyl cellulosesodium.

[6] The wound covering material according to any one of [1] to [5],

wherein the hydrogel contains 0.2 mass % or more and 1.8 mass % or less of fine fibrous celluloses having ionic substituents.

[7] The wound covering material according to any one of [1] to [6],

wherein, in the fine fibrous celluloses having ionic substituents, the amount of ionic substituents introduced per unit mass of fine fibrous celluloses is 0.10 mmol/g or more and 5.20 mmol/g or less.

[8] A method of producing a wound covering material containing at least a hydrogel, including

a process of obtaining a hydrogel using a hydrogel composition containing hydrophilic polymers and fine fibrous celluloses having ionic substituents.

[9] The method of producing a wound covering material according to [8],

wherein the hydrogel composition is obtained by mixing a hydrophilic polymer aqueous solution and a water dispersion solution containing fine fibrous celluloses having ionic substituents.

[10] The method of producing a wound covering material according to [8] or [9],

wherein radiation is emitted to the hydrogel composition, and hydrophilic polymers are crosslinked to form a hydrogel.

[11] The method of producing a wound covering material according to [9] or [10],

wherein the hydrophilic polymer aqueous solution contains 10 mass % or more and 90 mass % or less of hydrophilic polymers.

[12] The method of producing a wound covering material according to any one of [9] to [11],

wherein the water dispersion solution containing fine fibrous celluloses having ionic substituents contains 0.1 mass % or more and 15 mass % or less of fine fibrous celluloses having ionic substituents.

### EXAMPLES

Hereinafter, features of the present invention will be descried in more detail with reference to examples and comparative examples. The materials, amounts used, ratios, treatment contents, treatment procedures and the like shown in the following examples can be appropriately changed without departing from the spirit and scope of the present invention. Therefore, the scope of the present invention should not be construed as limited to the following specific examples.

### Production Example 1

<Phosphoric Oxidation Treatment>

As a raw material pulp, needle bleached kraft pulp (commercially available from Oji Paper Co., Ltd.) (a sheet form with a solid content of 93 mass % and a basis weight of 208 g/m², and the Canadian standard freeness (CSF) measured according to JIS P 8121-2: 2012 after dissociation was 700 mL) was used. A phosphoric oxidation treatment was performed on the raw material pulp as follows. First, an aqueous solution in which ammonium dihydrogen phosphate and urea were mixed was added to 100 parts by mass (absolute dry mass) of the raw material pulp to prepare 45 parts by mass of ammonium dihydrogen phosphate, 120 parts by mass of urea, and 150 parts by mass of water, and thereby a chemical-impregnated pulp was obtained. Next, the obtained chemical-impregnated pulp was heated in a hot air dryer at 165° C. for 200 seconds, and a phosphorus oxoacid group was introduced into the cellulose in the pulp to obtain a phosphoric-oxo oxidized pulp.

<Washing Treatment>

Next, a washing treatment was performed on the obtained phosphoric-oxo oxidized pulp. The washing treatment was performed by repeating an operation in which a pulp dispersion solution obtained by pouring 10 L deionized water to 100 g (absolute dry mass) of the phosphoric-oxo oxidized pulp was stirred so that the pulp was uniformly dispersed and then filtered and dehydrated. A time point at which the electric conductivity of the filtrate was 100 μS/cm or less was a washing end point.

<Neutralization Treatment>

The phosphoric oxidation treatment and the washing treatment were additionally performed on the phosphoric-oxo oxidized pulp after washing once in that order. Next, a neutralization treatment was performed on the phosphoric-oxo oxidized pulp after washing as follows. First, the phosphoric-oxo oxidized pulp after washing was diluted with 10 L deionized water and then a 1 N sodium hydroxide aqueous solution was added little by little with stirring, and thereby a phosphoric-oxo oxidized pulp slurry having a pH of 12 or more and 13 or less was obtained. Next, the phosphoric-oxo oxidized pulp slurry was dehydrated to obtain a neutralized phosphoric-oxo oxidized pulp.

An infrared absorption spectrum of the phosphoric-oxo oxidized pulp obtained in this manner was measured using FT-IR. As a result, absorption based on P═O of the phosphorus oxoacid group was observed around 1,230 cm⁻¹, and it was confirmed that the phosphorus oxoacid group was added to the pulp. In addition, when the obtained phosphoric-oxo oxidized pulp was tested, and analyzed using an X-ray diffractometer, typical peaks were confirmed at two positions where 20 was around 14° or more and 17° or less and 20 was around 22° or more and 23° or less, and it was confirmed that the pulp had a cellulose type I crystal.

<Defibration Treatment>

Deionized water was added to the obtained phosphoric-oxo oxidized pulp to prepare a slurry having a solid content concentration of 2 mass %. This slurry was treated twice using a wet atomizing device (Starburst commercially available from Sugino Machine Ltd.) at a pressure of 200 MPa to obtain a fine fibrous cellulose-containing dispersion solution having a solid content concentration of 2 mass %. The fine fibrous cellulose contained in the fine fibrous cellulose-containing dispersion solution obtained in Production Example 1 was used as P-CNF in examples to be described below.

It was confirmed by X-ray diffraction that the fine fibrous cellulose of Production Example 1 maintained a cellulose type I crystal. In addition, the fiber width of the fine fibrous cellulose of Production Example 1 measured using a transmission electron microscope was 3 nm to 5 nm. Here, the amount of phosphoric acid groups (first dissociated acid amount, strongly acidic group amount) measured in the method to be described below was 1.45 mmol/g. Here, the total amount of dissociated acids was 2.45 mmol/g.

<Measurement of Amount of Substituent>

The amount of phosphorus oxoacid groups contained in the P-CNF was measured by treating a fine fibrous cellulose-containing slurry prepared by diluting a fine fibrous cellulose-containing dispersion solution containing target fine fibrous celluloses with deionized water so that the content was 0.2 mass % with an ion exchange resin and then performing titration using an alkali.

The treatment with an ion exchange resin was performed by adding a strongly acidic cation exchange resin (Amberjet 1024; conditioning agent commercially available from Organo Corporation) with a volume of 1/10 to the fine fibrous cellulose-containing slurry, shaking for 1 hour, and then pouring it onto a mesh having an opening of 90 μm, and separating the resin and the slurry.

In addition, titration using an alkali was performed by measuring the change in the value of pH indicated by the slurry while adding 10 μL of a 0.1 N sodium hydroxide aqueous solution to the fine fibrous cellulose-containing slurry treated with the ion exchange resin every 5 seconds. Here, titration was performed while blowing nitrogen gas into the slurry from 15 minutes before titration started. In this neutralization titration, as shown in FIG. 1, in the curve plotting the pH measured with respect to the amount of an alkali added, two points at which the increment (the differential value of the pH with respect to the amount of an alkali dropped) become a maximum were observed. Among these, the maximum point of the increment obtained first when the alkali was added is called a first end point, and the maximum point of the increment obtained next is called a second end point (FIG. 1). The amount of an alkali required from the titration start to the first end point was the same as the amount of the first dissociated acids in the slurry used for titration. In addition, the amount of an alkali required from the titration start to the second end point was the same as the total amount of dissociated acids in the slurry used for titration. Here, the value obtained by dividing the amount of an alkali (mmol) required from the titration start to the first end point by the solid content (g) in the slurry to be titrated was defined as an amount of phosphorus oxoacid groups (first dissociated acid amount) (mmol/g). In addition, the value obtained by dividing the amount of an alkali (mmol) required from the titration start to the second end point by the solid content (g) in the slurry to be titrated was defined as a total amount of dissociated acids (mmol/g).

### Production Example 2

<TEMPO Oxidation Treatment>

As a raw material pulp, needle bleached kraft pulp (commercially available from Oji Paper Co., Ltd.) (a sheet form with a solid content of 93 mass % and a basis weight of 208 g/m², and the Canadian standard freeness (CSF) measured according to JIS P 8121-2: 2012 after dissociation was 700 mL) was used. A TEMPO oxidation treatment was performed on the raw material pulp as follows.

First, 100 parts by mass (dry mass) of the raw material pulp, 1.6 parts by mass of TEMPO (2,2,6,6-tetramethylpiperidin-1-oxyl), and 10 parts by mass of sodium bromide were dispersed in 10,000 parts by mass of water. Next, a 13 mass % sodium hypochlorite aqueous solution was added to 1.0 g of the pulp so that the concentration was 10 mmol, and the reaction was started. During the reaction, a 0.5 M sodium hydroxide aqueous solution was added dropwise, the pH was kept at 10 or more and 10.5 or less, and the reaction was considered to be completed when no change was observed in the pH.

<Washing Treatment>

Next, a washing treatment was performed on the obtained TEMPO oxidation pulp. The washing treatment was performed by repeating an operation in which the pulp slurry after TEMPO oxidation was dehydrated to obtain a dehydrated sheet, and 5,000 parts by mass of deionized water was then poured, stirring and uniform dispersion were performed, and filtering and dehydration were then performed. A time point at which the electric conductivity of the filtrate was 100 μS/cm or less was a washing end point.

In addition, when the obtained TEMPO oxidation pulp was tested and analyzed using an X-ray diffractometer, typical peaks were confirmed at two positions where 20 was around 14° or more and 17° or less and 20 was around 22° or more and 23° or less, and it was confirmed that the pulp had a cellulose type I crystal.

<Defibration Treatment>

Deionized water was added to the obtained TEMPO oxidation pulp to prepare a slurry having a solid content concentration of 2 mass %. This slurry was treated twice using a wet atomizing device (Starburst commercially available from Sugino Machine Ltd.) at a pressure of 200 MPa to obtain a fine fibrous cellulose-containing dispersion solution having a solid content concentration of 2 mass %. The fine fibrous cellulose contained in the fine fibrous cellulose-containing dispersion solution obtained in Production Example 2 was used as C-CNF in examples to be described below.

It was confirmed by X-ray diffraction that the fine fibrous cellulose of Production Example 2 maintained a cellulose type I crystal. In addition, the fiber width of the fine fibrous cellulose of Production Example 2 measured using a transmission electron microscope was 3 nm to 5 nm. Here, the amount of carboxyl groups (first dissociated acid amount, strongly acidic group amount) measured in the method to be described below was 1.80 mmol/g.

<Measurement of Amount of Substituent>

The amount of carboxyl groups contained in the C-CNF was measured by treating a fine fibrous cellulose-containing slurry prepared by diluting a fine fibrous cellulose-containing dispersion solution containing target fine fibrous celluloses with deionized water so that the content was 0.2 mass % with an ion exchange resin and then performing titration using an alkali.

The treatment with an ion exchange resin was performed by adding a strongly acidic cation exchange resin (Amberj et 1024; conditioning agent commercially available from Organo Corporation) with a volume of 1/10 to the fine fibrous cellulose-containing slurry, shaking for 1 hour, and then pouring it onto a mesh having an opening of 90 μm, and separating the resin and the slurry.

In addition, titration using an alkali was performed by measuring the change in the value of pH indicated by the slurry while adding 10 μL of a 0.1 N sodium hydroxide aqueous solution to the fine fibrous cellulose-containing slurry treated with the ion exchange resin every 5 seconds. Here, titration was performed while blowing nitrogen gas into the slurry from 15 minutes before titration started. In this neutralization titration, as shown in FIG. 2, in the curve plotting the pH measured with respect to the amount of an alkali added, one point at which the increment (the differential value of the pH with respect to the amount of an alkali dropped) become a maximum was observed. The increment maximum point is called a first end point (FIG. 2). Here, the region from the titration start to the first end point in FIG. 2 is called a first region. The amount of an alkali required for the first region is the same as the amount of carboxyl groups in the slurry use for titration. Then, the amount of carboxyl groups introduced (mmol/g) was calculated by dividing the amount of an alkali (mmol) required in the first region of the titration curve by the solid content (g) in the slurry to be titrated.

### Production Example 3

### (Sulfur Oxo Oxidation Treatment)

As a raw material pulp, needle bleached kraft pulp (commercially available from Oji Paper Co., Ltd.) (a sheet form with a solid content of 93 mass % and a basis weight of 245 g/m², and the Canadian standard freeness (CSF) measured according to JIS P 8121-2: 2012 after dissociation was 700 mL) was used. A sulfur oxo oxidation treatment was performed on the raw material pulp as follows. First, an aqueous solution in which amidosulfate and urea were mixed was added to 100 parts by mass (absolute dry mass) of the raw material pulp to prepare 38 parts by mass of amidosulfate, 120 parts by mass of urea, and 150 parts by mass of water, and thereby a chemical-impregnated pulp was obtained. Next, the obtained chemical-impregnated pulp was heated in a hot air dryer at 165° C. for 19 minutes, and a sulfate group was introduced into the cellulose in the pulp to obtain a sulfur-oxo oxidized pulp.

<Washing Treatment>

Next, a washing treatment was performed on the obtained sulfur-oxo oxidized pulp. The washing treatment was performed by repeating an operation in which a pulp dispersion solution obtained by pouring 10 L deionized water to 100 g (absolute dry mass) of the sulfur-oxo oxidized pulp was stirred so that the pulp was uniformly dispersed and then filtered and dehydrated. A time point at which the electric conductivity of the filtrate was 100 μS/cm or less was a washing end point.

Next, a neutralization treatment was performed on the sulfur-oxo oxidized pulp after washing was follows. First, the sulfur-oxo oxidized pulp after washing was diluted with 10 L deionized water, a 1 N sodium hydroxide aqueous solution was then added little by little with stirring, and thereby a sulfur-oxo oxidized pulp slurry having a pH of 12 or more and 13 or less was obtained. Next, the sulfur-oxo oxidized pulp slurry was dehydrated to obtain a neutralized sulfur-oxo oxidized pulp. Next, the washing treatment was performed on the neutralized sulfur-oxo oxidized pulp to obtain a sulfur-oxo oxidized pulp (neutralized once)

The obtained sulfur-oxo oxidized pulp was additionally subjected to the neutralization treatment and the washing treatment four times to obtain a sulfur-oxo oxidized pulp (neutralized five times).

<Defibration Treatment>

Deionized water was added to the obtained sulfur-oxo oxidized pulp (neutralized five times) and then stirred to prepare a slurry having a solid content concentration of 0.5 mass %. This slurry was defibrated using a defibration treatment device (high-speed rotary defibration treatment device CLEAMIX 2.2S commercially available from M Technique Co., Ltd.) under conditions of 21,500 rpm for 30 minutes, and thereby a dispersion solution containing fine fibrous celluloses having a fiber width of 3 nm to 5 nm was obtained. The fine fibrous cellulose contained in the fine fibrous cellulose-containing dispersion solution obtained in Production Example 3 was used as S-CNF in examples to be described below. Here, the amount of sulfur oxoacid groups measured by the method to be described below was 1.20 mmol/g.

<Measurement of Amount of Substituent>

For the amount of sulfur oxoacid groups contained in S-CNF, a sample after freeze-drying and crushing treatments was pressurized, heated, and decomposed with nitric acid in a closed container, and diluted appropriately, and the amount of sulfur was measured through ICP-OES. The value calculated by performing division by the absolute dry mass of the test fine fibrous celluloses was defined as an amount of sulfur oxoacid groups (mmol/g).

### Example 1

60 parts by mass (the mass of the P-CNF water dispersion solution was 1.2 g) of the P-CNF water dispersion solution obtained in Production Example 1 was added to 40 parts by mass (the mass of the PVA aqueous solution was 0.8 g) of a PVA aqueous solution (PVA (commercially available from Wako Pure Chemical Industries, Ltd.) a 20 mass % (w/w) aqueous solution prepared using (“160-08295,” degree of saponification: 72 mol % to 82 mol %, average degree of polymerization: about 2, 000); hereinafter the same applies), the mixture was stirred with a microspatula, and thus a hydrogel composition was obtained. The hydrogel composition was spread thinly on a polystyrene petri dish so that the thickness was 1 mm. Next, using an electro-curtain type electron beam radiation device EC250/30/90L (commercially available from Iwasaki Electric Co., Ltd.), a crosslink reaction was caused by emitting electron beams of 50 kGy at an acceleration voltage of 250 kV under a nitrogen atmosphere. Then, deionized water was added to the obtained hydrogel (hereinafter referred to as a PVA-P-CNF gel), and a PVA-P-CNF gel was peeled off from the polystyrene petri dish with a microspatula. The peeled PVA-P-CNF gel was washed with deionized water five times, and the unreacted PVA was removed.

### Example 2

A hydrogel (hereinafter referred to as a PVA-C-CNF gel) was prepared in the same manner as in Example 1 except that a hydrogel composition obtained by adding 60 parts by mass of the C-CNF water dispersion solution (the mass of the C-CNF water dispersion solution was 1.2 g) obtained in Production Example 2 to 40 parts by mass of the PVA aqueous solution (the mass of the PVA aqueous solution was 0.8 g) and performing stirring and mixing was obtained.

### Example 3

A hydrogel (hereinafter referred to as a PVA-S-CNF gel) was prepared in the same manner as in Example 1 except that a hydrogel composition obtained by adding 60 parts by mass (the mass of the S-CNF water dispersion solution was 1.2 g) of the S-CNF water dispersion solution obtained in Production Example 3 to 40 parts by mass of the PVA aqueous solution (the mass of the PVA aqueous solution was 0.8 g), and performing stirring and mixing was obtained.

### Comparative Example 1

A hydrogel (hereinafter referred to as a PVA gel) was obtained in the same manner as in Example 1 except that only 40 parts by mass of the PVA aqueous solution (the mass of the PVA aqueous solution was 0.8 g) was used.

The water retention rates of the hydrogels of Examples 1 to 3 and Comparative Example 1 were measured as follows, and the results of the relative water retention rate (%) when the water retention rate of Comparative Example 1 was 100% are shown in the following Table 1.

(Measurement of Water Retention Rate)

The water retention rate was measured according to the following procedures. Here, a higher water retention rate indicates a larger water retention capacity.

(1) A hydrogel was air-dried at room temperature (20° C.) for 72 hours or longer, and the dry mass was measured (W0).
(2) A dried gel was put into deionized water in an amount 150 times the mass of the dried gel or more, and water was absorbed for 24 hours.
(3) The water-containing gel in which water was absorbed was taken out, excess water was removed on a filter paper (No. 5C (commercially available from Advantec)) for 10 seconds, and the mass was then measured (W1).
(4) The water retention rate WR (%) was determined by the following formula.

WR(%)=(W1−W0)/(W0)×100

TABLE 1

| | | | | Comparative |
|---|---|---|---|---|
| | Example 1 | Example 2 | Example 3 | Example 1 |
| Relative water | 387 | 484 | 443 | 100 |
| retention rate (%) | | | | |

As can be understood from the results in Table 1, the relative water retention rates of the hydrogels of Example 1, Example 2 and Example 3 were three times that of Comparative Example 1 or more, and the hydrogels of Example 1, Example 2 and Example 3 had a significantly larger water retention capacity than the hydrogel of Comparative Example 1. This is because the hydrogels of Example 1, Example 2 and Example 3 contained fine fibrous celluloses having ionic substituents. In addition, since the hydrogels of Example 1, Example 2 and Example 3 contained fine fibrous celluloses having ionic substituents, they had high strength and were not easily broken, and had favorable handling properties when pinched with, for example, tweezers, and were easily cut to a predetermined size.

### Example 4

60 parts by mass of the P-CNF water dispersion solution was added to 40 parts by mass of the PVA aqueous solution, and the mixture was stirred and mixed to obtain a hydrogel composition. The proportions of PVA and P-CNF in the hydrogel composition were the same as in the hydrogel composition in Example 1. 1 g of the hydrogel composition was placed in a 6-well plate, and shaped into a form having a diameter of about 2 cm (25(p). Next, using an electro-curtain type electron beam radiation device EC250/30/90L (commercially available from Iwasaki Electric Co., Ltd.), a crosslink reaction was caused by emitting electron beams of 50 kGy at an acceleration voltage of 250 kV under a nitrogen atmosphere. Then, the obtained hydrogel (hereinafter referred to as a PVA-P-CNF gel) was peeled off from the plate with a microspatula, pinched with tweezers, and washed with deionized water, and the unreacted PVA was removed. Then, the sample was stored in deionized water and refrigerated.

### Comparative Example 2

A hydrogel was obtained in the same manner as in Example 4 except that only a PVA aqueous solution was used.

### Reference Example 1

A medical gauze type I (4 layers) was cut into 20 mm squares and used as Reference Example 1.

The wound healing effect was confirmed using the hydrogels of Example 4 and Comparative Example 2, and the gauze of Reference Example 1.

(Wound Healing Test)

(1) Animals used
(1.1) Animal species, lineage and sex: rat, Slc: Wistar, SPF, male
(1.2) Supply source: commercially available from Japan SLC, Inc. (Hamamatsu City, Shizuoka Prefecture)
(1.3) Age and number of rats used:

Age at arrival: 15 weeks; at arrival: 19 rats

Age when the test was performed: 16 to 17 weeks

### (1.4) Quarantine and Acclimatization Method

(a) A quarantine/acclimatization period was set for 7 days after arrival.
(b) During the quarantine/acclimatization period, the general state was observed once a day, and the body weight was measured the day after arrival of the animals and the end day of quarantine/acclimatization.
(c) Healthy animals that showed good growth in the general state and body weight performance during the quarantine/acclimatization period were used in the test.

### (1.5) Grouping Method

Based on the body weight at the end day of acclimatization, 15 rats were selected from all rats, excluding two heavy rats and two light rats, and divided into three groups according to a completely random sampling method using a computer so that the average body weights of the groups were the same.

### (2) Group Composition and Treatment Method

### (2.1) Group Composition, Test Substance, Administration Dose, Treatment and Number of Cases

TABLE 2

| | | Size of covering | Application | Number |
|---|---|---|---|---|
| Test group | Test substance | material | period | of cases |
| A1 | Reference | 20 mm × 20 mm | For 8 days | 5 |
| | Example 1 | | | |
| A2 | Comparative | Equivalent to a | For 8 days | 5 |
| | Example 2 | diameter of 25 mm | | |
| A3 | Example 4 | Equivalent to a | For 8 days | 5 |
| | | diameter of 25 mm | | |

### (2.2) Administration Method of Control Substance and Test Substance

(a) Administration frequency and administration period: one covering material was applied daily for 8 days.
(b) Administration method: a covering material was applied so that the wound site was completely covered, and a waterproof film (commercially available from Nichiban Co., Ltd.) was applied so that the rats could not peel off the covering material. After the waterproof film was fixed with an elastic tape (Tear light tape, commercially available from Mueller Japan Co., Ltd.), rat jackets (clothing) were put on.

### (3) Operation Items

### (3.1) State Observation

During the test substance application period, the state was observed daily in all cases.

### (3.2) Measurement of Body Weight

The body weight was measured daily from the first day of test substance application (the day of wound preparation) to the ninth day of application.

### (3.3) Preparation of Skin Wound

Rats were anesthetized to create a defect wound with a diameter of 15 mm.

(3.4) Imaging and area measurement of skin wound
(a) The wound site was imaged using a digital camera (Power Shot S3 IS, commercially available from Canon Inc.) before the test substance was applied on the 1st to 8th days after the test substance was applied (the day of wound preparation) and on the 9th day from the day of wound preparation.
(b) The wound site was marked in the captured wound digital image using image software (Photo Studio 4 for Canon, ArcSoft. Inc.) and the area (cm²) was then measured by ImageJ (Ver. 10.2).
(c) The area of the wound part on the start day of test substance application was set as 100%, and the area ratio (%) on each measurement day was calculated.

### (4) Statistical Method

(a) Each measured value was expressed as an average value±standard error for each group.
(b) For comparison between the groups of the areas of the wound parts and the area ratios on the day of test substance application, the Tukey's multiple comparison test was performed for three groups Al to A3.
(c) StatLightR (commercially available from Yukms Co., Ltd.) was used for statistical analysis, and the significance level was set to less than 5%.

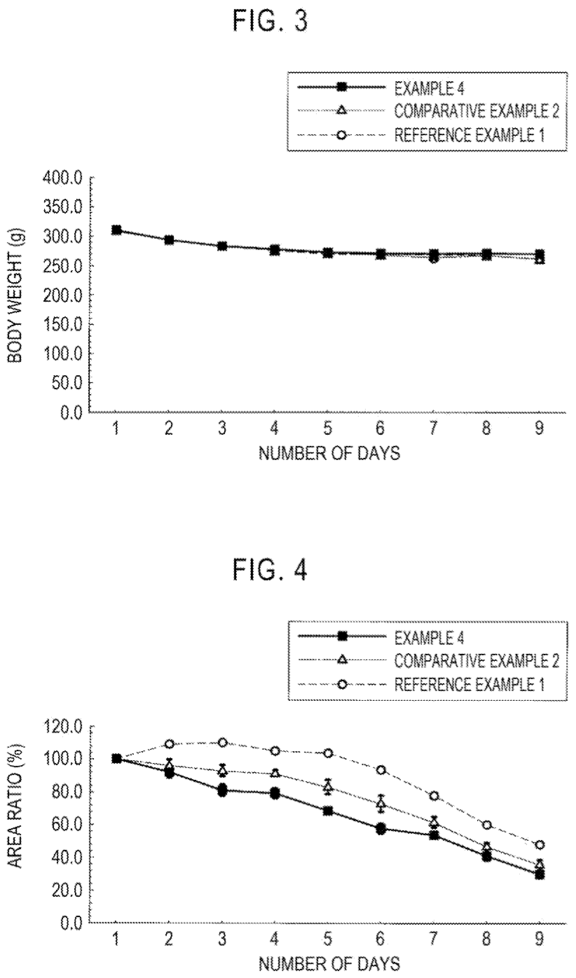

FIG. 3 shows the changes in the body weights of the rats when the PVA-P-CNF gel of Example 4, the hydrogel of Comparative Example 2 and the gauze of Reference Example 1 were applied for 9 days as the covering material after wounds were created on the backs of the rats. All application groups showed a gradual decrease in the body weight from the day after wound creation, but regarding the body weights of the groups on each measurement day to which the PVA-P-CNF gel of Example 4, the hydrogel of Comparative Example 2 and the gauze of Reference Example 1 were applied, no significant change was observed on all of the measurement days, and no significant abnormalities were observed in the general state during the application period. Here, the adhesiveness and peelability of the covering material with respect to the rat skin were favorable.

FIG. 4 shows the area ratio when the wound area of Day1 from the day of wound preparation (Day1) to the final day (Day9) when the PVA-P-CNF gel of Example 4, the hydrogel of Comparative Example 2 and the gauze of Reference Example 1 were applied as the covering material was set as 100%. The average values of the wound areas at the preparation of groups to which the PVA-P-CNF gel of Example 4, the hydrogel of Comparative Example 2 and the gauze of Reference Example 1 were applied as the covering material were 2.10 cm², 1.85 cm² and 2.08 cm², respectively. Regarding the area ratio when the wound area of Day1 was set as 100%, in the groups to which the PVA-P-CNF gel of Example 4, the hydrogel of Comparative Example 2 and the gauze of Reference Example 1 were applied, the area ratios decreased to 30.1%, 35.8% and 47.4%, respectively, on Day9. Regarding the wound area ratio of the group to which the gauze of Reference Example 1 was applied, in the groups to which the PVA-P-CNF gel of Example 4 and the hydrogel of Comparative Example 2 were applied, a significant decrease was observed from Day2 to Day9, and from Day3 to Day6, a significant decrease in the wound area ratio was observed in the group to which the PVA-P-CNF gel of Example 4 was applied as compared with the group to which the hydrogel of Comparative Example 2 was applied.

1. A wound covering material comprising at least a hydrogel, wherein the hydrogel contains fine fibrous celluloses having ionic substituents.

2. The wound covering material according to claim 1, wherein the ionic substituent is one or more anionic groups selected from the group consisting of a phosphorus oxoacid group, a substituent derived from a phosphorus oxoacid group, a carboxyl group, a substituent derived from a carboxyl group, a sulfur oxoacid group, and a substituent derived from a sulfur oxoacid group.

3. The wound covering material according to claim 1, wherein the fine fibrous celluloses have an average fiber width of 1,000 nm or less.

4. The wound covering material according to claim 1, wherein the hydrogel is formed of crosslinked hydrophilic polymers.

5. The wound covering material according to claim 4, wherein the hydrophilic polymer is one or more selected from the group consisting of polyvinyl alcohol, polyvinylpyrrolidone, and carboxymethyl cellulose sodium.

6. The wound covering material according to claim 1, wherein the hydrogel contains 0.2 mass % or more and 1.8 mass % or less of fine fibrous celluloses having ionic substituents.

7. The wound covering material according to claim 1, wherein, in the fine fibrous celluloses having ionic substituents, the amount of ionic substituents introduced per unit mass of fine fibrous celluloses is 0.10 mmol/g or more and 5.20 mmol/g or less.

8. A method of producing a wound covering material including at least a hydrogel, comprising
a process of obtaining a hydrogel using a hydrogel composition containing hydrophilic polymers and fine fibrous celluloses having ionic substituents.

9. The method of producing a wound covering material according to claim 8, wherein the hydrogel composition is obtained by mixing a hydrophilic polymer aqueous solution and a water dispersion solution containing fine fibrous celluloses having ionic substituents.

10. The method of producing a wound covering material according to claim 8, wherein radiation is emitted to the hydrogel composition, and hydrophilic polymers are crosslinked to form a hydrogel.

11. The method of producing a wound covering material according to claim 9, wherein the hydrophilic polymer aqueous solution contains 10 mass % or more and 90 mass % or less of hydrophilic polymers.

12. The method of producing a wound covering material according to claim 9, wherein the water dispersion solution containing fine fibrous celluloses having ionic substituents contains 0.1 mass % or more and 15 mass % or less of fine fibrous celluloses having ionic substituents.
